SEQUENCE LISTING (8 sequences)

SEQ ID NO: 1 - DNA, 1377 bp, Stevia rebaudiana bertoni
atggaaaataaaacggagaccaccgttcgccggcgccggagaataatattattcccggtaccatttcaaggccacattaacccaattcttcagctagccaatgtgttgtactctaaaggattcagtatcaccatctttcacaccaacttcaacaaacccaaaacatctaattaccctcacttcactttcagattcatcctcgacaacgacccacaagacgaacgcatttccaatctaccgactcatggtccgctcgctggtatgcggattccgattatcaacgaacacggagctgacgaattacgacgcgaactggaactgttgatgttagcttctgaagaagatgaagaggtatcgtgtttaatcacggatgctctttggtacttcgcgcaatctgttgctgacagtcttaacctccgacggcttgttttgatgacaagcagcttgtttaattttcatgcacatgtttcacttcctcagtttgatgagcttggttacctcgatcctgatgacaaaacccgtttggaagaacaagcgagtgggtttcctatgctaaaagtgaaagacatcaagtctgcgtattcgaactggcaaatactcaaagagatattagggaagatgataaaacaaacaaaagcatcttcaggagtcatctggaactcatttaaggaactcgaagagtctgagctcgaaactgttatccgtgagatcccggctccaagtttcttgataccactccccaagcatttgacagcctcttccagcagcttactagaccacgatcgaaccgtttttcaatggttagaccaacaaccgccaagttcggtactgtatgttagttttggtagtactagtgaagtggatgagaaagatttcttggaaatagctcgtgggttggttgatagcaagcagtcgtttttatgggtggttcgacctgggtttgtcaagggttcgacgtgggtcgaaccgttgccagatgggttcttgggtgaaagaggacgtattgtgaaatgggttccacagcaagaagtgctagctcatggagcaataggcgcattctggactcatagcggatggaactctacgttggaaagcgtttgtgaaggtgttcctatgattttctcggattttgggctcgatcaaccgttgaatgctagatacatgagtgatgttttgaaggtaggggtgtatttggaaaatgggtgggaaagaggagagatagcaaatgcaataagaagagttatggtggatgaagaaggagaatacattagacagaatgcaagagttttgaaacaaaaggcagatgtttctttgatgaagggtggttcgtcttacgaatcattagagtctctagtttcttacatttcatcgttgtaa

SEQ ID NO: 2 - protein, 458 aa, Stevia rebaudiana bertoni
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 3 - DNA, 1377 bp, Artificial Sequence
  optimized sequence derived from Stevia rebaudiana bertoni
atggagaataaaacggaaaccaccgttcgtcgccgtcgtcgcattatcctgttcccggtgccgttccagggtcacattaacccgatcctgcagctggccaatgtgctgtatagcaaaggctttagcatcaccattttccacaccaacttcaataaaccgaaaacgagcaattacccgcactttaccttccgcttcattctggacaatgacccgcaagacgagcgtatcagcaacctgccgacccatggcccgctggcgggtatgcgtatcccgatcatcaatgaacatggtgcggacgaactgcgtcgcgaactggaactgctgatgctggcgagcgaagaagatgaagaagtgagctgcctgattaccgatgcgctgtggtattttgcgcagagcgttgcggatagcctgaacctgcgccgtctggttctgatgaccagcagcctgttcaatttccatgcccacgttagcctgccgcaattcgatgagctgggctatctggacccggatgataaaacccgcctggaagagcaagcgagcggctttccgatgctgaaagttaaagacattaaaagcgcgtacagcaactggcagatcctgaaagaaattctgggcaaaatgatcaaacaaaccaaagccagcagcggtgttatttggaacagcttcaaagaactggaggagagcgagctggagacggttatccgtgagatcccggccccgagctttctgattccgctgccgaaacatctgaccgcgagcagcagcagcctgctggaccacgaccgcaccgtttttcagtggctggatcaacagccgccgagcagcgttctgtatgttagctttggcagcaccagcgaagttgacgagaaagactttctggaaatcgcccgtggtctggtggacagcaaacagagcttcctgtgggtggtgcgtccgggctttgttaaaggtagcacctgggtggagccgctgccggacggtttcctgggcgaacgtggccgcatcgttaaatgggttccgcagcaggaagttctggcgcatggcgcgattggcgcgttttggacccatagcggctggaatagcacgctggaaagcgtttgcgagggcgtgccgatgatttttagcgattttggcctggaccaaccgctgaacgcccgctatatgagcgatgttctgaaagtgggtgtttacctggaaaacggctgggagcgcggtgaaattgcgaatgccattcgtcgtgtgatggtggatgaggaaggcgagtatattcgtcaaaatgcgcgcgtgctgaaacaaaaagcggacgtgagcctgatgaaaggtggtagcagctacgaaagcctggagagcctggtgagctatattagcagcctgtaa

SEQ ID NO: 4 - DNA, 1380 bp, Hordeum vulgare subsp. vulgare
atggacggcaactcctcctcctcgccgctgcacgtggtgatctgcccgtggctcgccttgggccacctgctgccgtgcctggacatcgccgagcgcctggcgtcgcgcggccaccgcgtctccttcgtctccacgccgcgcaacatcgcgcgcctcccgccgctccggcccgccgtggcgccgctcgtcgacttcgtcgcgctgccgctcccgcacgtcgacggcctccccgagggcgccgagtcgaccaacgacgtcccctacgacaagttcgagctccaccgcaaggccttcgacggcctcgccgcgcccttctcggagttcctgcgcgccgcgtgcgccgagggcgctggcagcaggcccgactggctcatcgtcgacaccttccaccactgggccgccgcggccgccgtcgaaaataaggttccatgcgtgatgcttctgctgggagccgcgaccgtgatcgcaggcttcgcccgaggtgtgtcggagcacgccgcggccgccgtcgggaaagagcgaccggcggcggaagcgccaagcttcgagacggagaggaggaagctgatgaccacccagaacgcatcggggatgacggtcgccgagcgctacttcctgacgctcatgaggagcgacctcgtggccatccggagctgcgccgagtgggagcccgagagcgtcgccgcgctcaccacgctcgcgggcaagccggtcgtccctctcggcctcctcccgccgtcgcccgagggaggccgcggcgtcagcaaggaggacgccgctgtgcgctggctcgacgcgcagccggccaagtcggtggtctacgtcgcgctcgggagcgaggtgccgctgcgcgccgagcaggtgcacgagctcgccctcgggctggagctctccggggcgcgcttcctctgggcgctgcggaagccgaccgacgcaccggacgcggccgtcctcccgccggggttcgaggagcgcacgcgcggccgcgggctggtggtgaccgggtgggttcctcagatcggcgtgctggcgcacggcgccgtggccgcgttcctgacgcactgcgggtggaactcgaccatcgaagggctgctgttcgggcacccgctcatcatgctgcccatctccagcgaccaggggcccaacgcgaggctcatggaggggaggaaggtcgggatgcaggtgccgagagacgaaagcgacggatcgttccgcagggaggacgtcgcggcgacggtgcgggccgtcgccgtggaggaagacggcaggagggtcttcacggccaacgccaagaagatgcaggagatcgtcgccgacggcgcttgccatgagaggtgcatcgacgggtttattcagcagctcagatcctacaaggcatga

SEQ ID NO: 5 - protein, 459 aa, Hordeum vulgare subsp. vulgare
MDGNSSSSPLHVVICPWLALGHLLPCLDIAERLASRGHRVSFVSTPRNIARLPPLRPAVAPLVDFVALPLPHVDGLPEGAESTNDVPYDKFELHRKAFDGLAAPFSEFLRAACAEGAGSRPDWLIVDTFHHWAAAAAVENKVPCVMLLLGAATVIAGFARGVSEHAAAAVGKERPAAEAPSFETERRKLMTTQNASGMTVAERYFLTLMRSDLVAIRSCAEWEPESVAALTTLAGKPVVPLGLLPPSPEGGRGVSKEDAAVRWLDAQPAKSVVYVALGSEVPLRAEQVHELALGLELSGARFLWALRKPTDAPDAAVLPPGFEERTRGRGLVVTGWVPQIGVLAHGAVAAFLTHCGWNSTIEGLLFGHPLIMLPISSDQGPNARLMEGRKVGMQVPRDESDGSFRREDVAATVRAVAVEEDGRRVFTANAKKMQEIVADGACHERCIDGFIQQLRSYKA

SEQ ID NO: 6 - DNA, 1383 bp, Artificial Sequence
  optimized sequence derived from Hordeum vulgare subsp. vulgare
atggacggtaactcttcttcttctccgctgcacgttgttatctgcccgtggctggctctgggtcacctgctgccgtgcctggacatcgctgaacgtctggcttctcgtggtcaccgtgtttctttcgtttctaccccgcgtaacatcgctcgtctgccgccgctgcgtccggctgttgctccgctggttgacttcgttgctctgccgctgccgcacgttgacggtctgccggaaggtgctgaatctaccaacgacgttccgtacgacaaattcgaactgcaccgtaaagctttcgacggtctggctgctccgttctctgaattcctgcgtgctgcttgcgctgaaggtgctggttctcgtccggactggctgatcgttgacaccttccaccactgggctgctgctgctgctgttgaaaacaaagttccgtgcgttatgctgctgctgggtgctgctaccgttatcgctggtttcgctcgtggtgtttctgaacacgctgctgctgctgttggtaaagaacgtccggctgctgaagctccgtctttcgaaaccgaacgtcgtaaactgatgaccacccagaacgcttctggtatgaccgttgctgaacgttacttcctgaccctgatgcgttctgacctggttgctatccgttcttgcgctgaatgggaaccggaatctgttgctgctctgaccaccctggctggtaaaccggttgttccgctgggtctgctgccgccgtctccggaaggtggtcgtggtgtttctaaagaagacgctgctgttcgttggctggacgctcagccggctaaatctgttgtttacgttgctctgggttctgaagttccgctgcgtgctgaacaggttcacgaactggctctgggtctggaactgtctggtgctcgtttcctgtgggctctgcgtaaaccgaccgacgctccggacgctgctgttctgccgccgggtttcgaagaacgtacccgtggtcgtggtctggttgttaccggttgggttccgcagatcggtgttctggctcacggtgctgttgctgctttcctgacccactgcggttggaactctaccatcgaaggtctgctgttcggtcacccgctgatcatgctgccgatctcttctgaccagggtccgaacgctcgtctgatggaaggtcgtaaagttggtatgcaggttccgcgtgacgaatctgacggttctttccgtcgtgaagacgttgctgctaccgttcgtgctgttgctgttgaagaagacggtcgtcgtgttttcaccgctaacgctaaaaaaatgcaggaaatcgttgctgacggtgcttgccacgaacgttgcatcgacggtttcatccagcagctgcgttcttacaaagcttaataa

SEQ ID NO: 7 - DNA, 2354 bp, Arabidopsis thaliana
aacgtaacgaagttctggctctgctgtctcgtgttgaagctaaaggtaaaggtatcctgcagcagaaccagatcatcgctgaattcgaagctctgccggaacagacccgtaaaaaactggaaggtggtccgttcttcgacctgctgaaatctacccaggaagctatcgttctgccgccgtgggttgctctggctgttcgtccgcgtccgggtgtttgggaatacctgcgtgttaacctgcacgctctggttgttgaagaactgcagccggctgaattcctgcacttcaaagaagaactggttgacggtgttaaaaacggtaacttcaccctggaactggacttcgaaccgttcaacgcttctatcccgcgtccgaccctgcacaaatacatcggtaacggtgttgacttcctgaaccgtcacctgtctgctaaactgttccacgacaaagaatctctgctgccgctgctgaaattcctgcgtctgcactctcaccagggtaaaaacctgatgctgtctgaaaaaatccagaacctgaacaccctgcagcacaccctgcgtaaagctgaagaatacctggctgaactgaaatctgaaaccctgtacgaagaattcgaagctaaattcgaagaaatcggtctggaacgtggttggggtgacaacgctgaacgtgttctggacatgatccgtctgctgctggacctgctggaagctccggacccgtgcaccctggaaaccttcctgggtcgtgttccgatggttttcaacgttgttatcctgtctccgcacggttacttcgctcaggacaacgttctgggttacccggacaccggtggtcaggttgtttacatcctggaccaggttcgtgctctggaaatcgaaatgctgcagcgtatcaaacagcagggtctgaacatcaaaccgcgtatcctgatcctgacccgtctgctgccggacgctgttggtaccacctgcggtgaacgtctggaacgtgtttacgactctgaatactgcgacatcctgcgtgttccgttccgtaccgaaaaaggtatcgttcgtaaatggatctctcgtttcgaagtttggccgtacctggaaacctacaccgaagacgctgctgttgaactgtctaaagaactgaacggtaaaccggacctgatcatcggtaactactctgacggtaacctggttgcttctctgctggctcacaaactgggtgttacccagtgcaccatcgctcacgctctggaaaaaaccaaatacccggactctgacatctactggaaaaaactggacgacaaataccacttctcttgccagttcaccgctgacatcttcgctatgaaccacaccgacttcatcatcacctctaccttccaggaaatcgctggttctaaagaaaccgttggtcagtacgaatctcacaccgctttcaccctgccgggtctgtaccgtgttgttcacggtatcgacgttttcgacccgaaattcaacatcgtttctccgggtgctgacatgtctatctacttcccgtacaccgaagaaaaacgtcgtctgaccaaattccactctgaaatcgaagaactgctgtactctgacgttgaaaacaaagaacacctgtgcgttctgaaagacaaaaaaaaaccgatcctgttcaccatggctcgtctggaccgtgttaaaaacctgtctggtctggttgaatggtacggtaaaaacacccgtctgcgtgaactggctaacctggttgttgttggtggtgaccgtcgtaaagaatctaaagacaacgaagaaaaagctgaaatgaaaaaaatgtacgacctgatcgaagaatacaaactgaacggtcagttccgttggatctcttctcagatggaccgtgttcgtaacggtgaactgtaccgttacatctgcgacaccaaaggtgctttcgttcagccggctctgtacgaagctttcggtctgaccgttgttgaagctatgacctgcggtctgccgaccttcgctacctgcaaaggtggtccggctgaaatcatcgttcacggtaaatctggtttccacatcgacccgtaccacggtgaccaggctgctgacaccctggctgacttcttcaccaaatgcaaagaagacccgtctcactgggacgaaatctctaaaggtggtctgcagcgtatcgaagaaaaatacacctggcagatctactctcagcgtctgctgaccctgaccggtgtttacggtttctggaaacacgtttctaacctggaccgtctggaagctcgtcgttacctggaaatgttctacgctctgaaataccgtccgctggctcaggctgttccgctggctcaggacgactaa

SEQ ID NO: 8 - protein, 808 aa, Arabidopsis thalia
MANAERMITRVHSQRERLNETLVSERNEVLALLSRVEAKGKGILQQNQIIAEFEALPEQTRKKLEGGPFFDLLKSTQEAIVLPPWVALAVRPRPGVWEYLRVNLHALVVEELQPAEFLHFKEELVDGVKNGNFTLELDFEPFNASIPRPTLHKYIGNGVDFLNRHLSAKLFHDKESLLPLLKFLRLHSHQGKNLMLSEKIQNLNTLQHTLRKAEEYLAELKSETLYEEFEAKFEEIGLERGWGDNAERVLDMIRLLLDLLEAPDPCTLETFLGRVPMVFNVVILSPHGYFAQDNVLGYPDTGGQVVYILDQVRALEIEMLQRIKQQGLNIKPRILILTRLLPDAVGTTCGERLERVYDSEYCDILRVPFRTEKGIVRKWISRFEVWPYLETYTEDAAVELSKELNGKPDLIIGNYSDGNLVASLLAHKLGVTQCTIAHALEKTKYPDSDIYWKKLDDKYHFSCQFTADIFAMNHTDFIITSTFQEIAGSKETVGQYESHTAFTLPGLYRVVHGIDVFDPKFNIVSPGADMSIYFPYTEEKRRLTKFHSEIEELLYSDVENKEHLCVLKDKKKPILFTMARLDRVKNLSGLVEWYGKNTRLRELANLVVVGGDRRKESKDNEEKAEMKKMYDLIEEYKLNGQFRWISSQMDRVRNGELYRYICDTKGAFVQPALYEAFGLTVVEAMTCGLPTFATCKGGPAEIIVHGKSGFHIDPYHGDQAADTLADFFTKCKEDPSHWDEISKGGLQRIEEKYTWQIYSQRLLTLTGVYGFWKHVSNLDRLEARRYLEMFYALKYRPLAQAVPLAQDD

(19) United States
(12) Patent Application Publication
(10) Pub. No.: US 2019/0203244 A1
(43) Pub. Date: Jul. 4, 2019

(54) PREPARATION OF REBAUDIOSIDE M IN A SINGLE REACTION VESSEL

(71) Applicant: PepsiCo, Inc., Purchase, NY (US)

(72) Inventors: Andrew ANDERSON, Chula Vista, CA (US); Alex CHU, Nanjing (CN); Chris DING, Nanjing (CN)

(21) Appl. No.: 15/932,218

(22) PCT Filed: Aug. 19, 2016
(86) PCT No.: PCT/US16/47772

§ 371 Date: Feb. 16, 2018

Related U.S. Application Data
(60) Provisional application No. 62/207,517, filed on Aug. 20, 2015.

(51) Int. Cl. C12P 19/56

(52) U.S. Cl. CPC C12P 19/56; A23L 2/60; C12Y 204/01013

(57) ABSTRACT

This disclosure describes a biotechnological method for producing rebaudioside M (Reb M), using recombinantly produced glucosyltransferases, which can be carried out in a single reaction vessel using two glucosyltransferases and a uridine diphosphate glucose (UDPG) regenerating system. The method can be used to produce the commercially desirable Reb M from rubusoside, stevioside, or rebaudioside A (Reb A), which are all naturally more abundant steviol glycosides but with commercially less desirable properties.

This disclosure incorporates by reference the contents of a 29 kb text file created on Aug. 19, 2016 and named “3711_273PC01_SL.txt,” which is the sequence listing for this application.

### TECHNICAL FIELD

This disclosure relates to methods of preparing rebaudioside compounds.

### BACKGROUND

Steviol glycosides belong to a group of diterpenoid with pentacylic steviol as the basic carbon core skeleton with different degree of glycosylation at C-13 hydroxyl and the C-19 ester groups. Differences in the degree and position of glycosylation causes the degree of sweetness and taste quality. Stevioside, which has two glucose units at C13 hydroxyl and one glucose at the C19 ester position, is the major steviol glycoside present in dry Stevia leaf (5-10% based on dry weight) and is 250-300 sweeter than sucrose but with some degree of undesirable aftertaste. Rebaudioside A, which has three glucose units attached at the C13 hydroxyl and one glucose unit at the C-19 ester group, is the second most abundant steviol glycoside (2-4% based on dry weight) and is 350-400 times sweeter than sucrose with no undesirable aftertaste. Rebaudioside D has one glucose unit attached to the C-19 ester group and has a better overall taste quality when compared to rebaudioside A. There is tremendous commercial interest to transform naturally more abundant steviol glycosides with less desirable commercial properties to steviol glycoside derivatives with more desirable commercial attributes.

### BRIEF DESCRIPTION OF THE DRAWINGS

FIG. 1. Schematic showing biotransformation of rebaudioside A to rebaudioside D and then to rebaudioside M.

FIG. 2. Schematic showing biotransformation of stevioside to rebaudioside E and then to rebaudioside M.

FIG. 3. Schematic showing biotransformation of rubusoside to rebaudioside E and then to rebaudioside M.

FIG. 4. Schematic showing biotransformation of either rubusoside, stevioside or rebaudioside to rebaudioside M.

FIG. 5A. LCMS spectrum of the crude reaction mixture from the biotransformation of stevioside to rebaudioside E. FIG. 5B. Mass Spectrum of the peak at 4:43.

FIG. 6A. LCMS of the crude reaction mixture from the biotransformation of rebaudioside A to rebaudioside D. FIG. 6B. Mass Spectrum of the peak at 4:43.

FIG. 7A. LCMS of the crude reaction mixture from the biotransformation of rebaudioside A to rebaudioside M in a single reaction vessel. FIG. 7B. Mass Spectrum of the peak at 4:90.

### DETAILED DESCRIPTION

This disclosure describes a highly robust biotechnological method for producing rebaudioside M (Reb M). The method uses recombinantly produced glucosyltransferases and is carried out in a single reaction vessel using two glucosyltransferases and a uridine diphosphate glucose (UDPG) regenerating system. The method produces high yields of the commercially desirable Reb M from rubusoside, stevioside, or rebaudioside A (Reb A), which are all naturally more abundant steviol glycosides but with commercially less desirable properties. The method is particularly advantageous because kinetic control is not necessary, and there is no need to use protective groups in order to achieve the specificity required to product Reb M.

The method can be carried out at a pH ranging from 7.0-9.0 (e.g., 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or 9.0) and at a temperature ranging from 20° C. to 37° C. (e.g., 20° C., 21° C., 22° C., 23° C., 24° C., 25° C., 26° C., 27 v, 28° C., 29° C., 30° C., 31° C., 32° C., 33° C., 34° C., 35° C., 36° C., or 37° C.). There are no problematic impurities produced, and no special purification conditions are required.

Glucosyltransferases

One glucosyltransferase is derived from barley (Hordeum vulgare subsp. vulgare) and has the ability to regio-selectively and stereoselectively transfer glucose from the donor UDPG (1) to rubusoside at both the C-13 and the C19 positions to convert rubusoside to Reb E, (2) to stevioside at the C-19 position to convert stevioside to Reb E and (3) to Reb A at the C19 position to convert Reb A to Reb D. Compared with other enzymes, such as UGT91D2 from Stevia as disclosed in WO2013/176738, or EUGT11 from rice (Oryza sativa, GenBank Accession No. AC133334) disclosed in WO2013/022989, which can only use stevioside as a substrate at 0.1 mM and 0.5 mM, with low conversion rates, this barley enzyme converts rubusoside and stevioside to Reb E and Reb A to Reb D at substrate concentrations of 2.4-5 mM with a near 100% conversion rate.

The other glucosyltransferase is UGT76G1 (SEQ ID NO:2), which is derived from Stevia rebaudiana subsp. Bertoni. UGT76G1 and has the ability (1) to catalyze the conversion of Reb D to Reb M and (2) to catalyze the simultaneous addition of two glucose units to Reb E, one at C-19 hydroxyl and one at C-13 ester group, respectively, to provide Reb M.

The two glucosyltransferases can be obtained from their natural sources, produced synthetically, or produced recombinantly. Examples of recombinant production in E. coli are provided in the examples below, but the enzymes can be produced using other microorganisms (e.g., Bacillus subtilis, Bacillus licheniformis, Bacillus amyloliquefaciens, Bacillus Stearothermophilus, Pichia pastoris, Saccharomyces cerevisiae, Kluyveromyces lactis, Aspergillus oryzae, Rhizomucor miehei, Aspergillus niger, Aspergillus awamori, Aspergillus nidulans, Fusarium oxysporum), cell lines (e.g., Drosophila S2 cells, Spodoptera Sf9 cells, CHO cells, COS cells, BHK cells, 293 cells, and Bowes cells), as well as using any of the other methods for recombinant production of proteins which are well known in the art. The glucosyltransferases need not be purified; that is, crude enzyme preparations can be used as demonstrated by the working examples, below.

UDPG Regenerating System

The reactions catalyzed by the glucosyltransferase enzymes described above use UDP-glucose as a glucosyl donor. The UDP-glucose can be regenerated in situ by including a UDPG regenerating system in the reaction. The UDPG regenerating system comprises (a) UDPG and (b) the UDPG recycling enzyme, sucrose synthase, which catalyzes the reaction between sucrose and UDP to provide UDP-glucose and fructose. The sucrose synthase can be purified from its natural source, produced synthetically, or produced recombinantly. The Examples below use a sucrose synthase from Arabidopsis thaliana (SEQ ID NO:8), which was produced recombinantly in E. coli; however, other sucrose synthases can be used, such as those obtained from corn, sorghum, barley, wheat, rice, or bamboo. As with the glucosyltransferases discussed above, the sucrose synthase can be produced in other produced using other microorganisms (e.g., Bacillus subtilis, Bacillus licheniformis, Bacillus amyloliquefaciens, Bacillus Stearothermophilus, Pichia pastoris, Saccharomyces cerevisiae, Kluyveromyces lactis, Aspergillus oryzae, Rhizomucor miehei, Aspergillus niger, Aspergillus awamori, Aspergillus nidulans, Fusarium oxysporum), cell lines (e.g., Drosophila S2 cells, Spodoptera Sf9 cells, CHO cells, COS cells, BHK cells, 293 cells, and Bowes cells), as well as using any of the other methods for recombinant production of proteins which are well known in the art.

Progress of the biotransformation can be monitored, for example, by HPLC, as described in the working examples, below, or by other methods such as liquid chromatography-mass spectroscopy (LCMS) or HPLC with an evaporative light scattering detector (HPLC-ELSD).

Methods of Producing Reb M

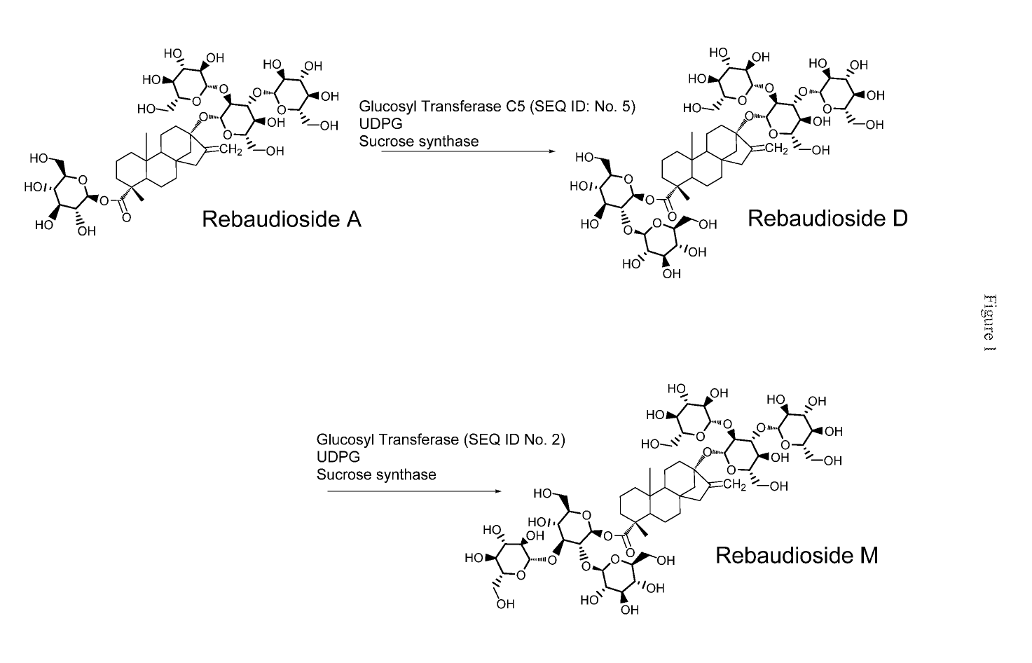

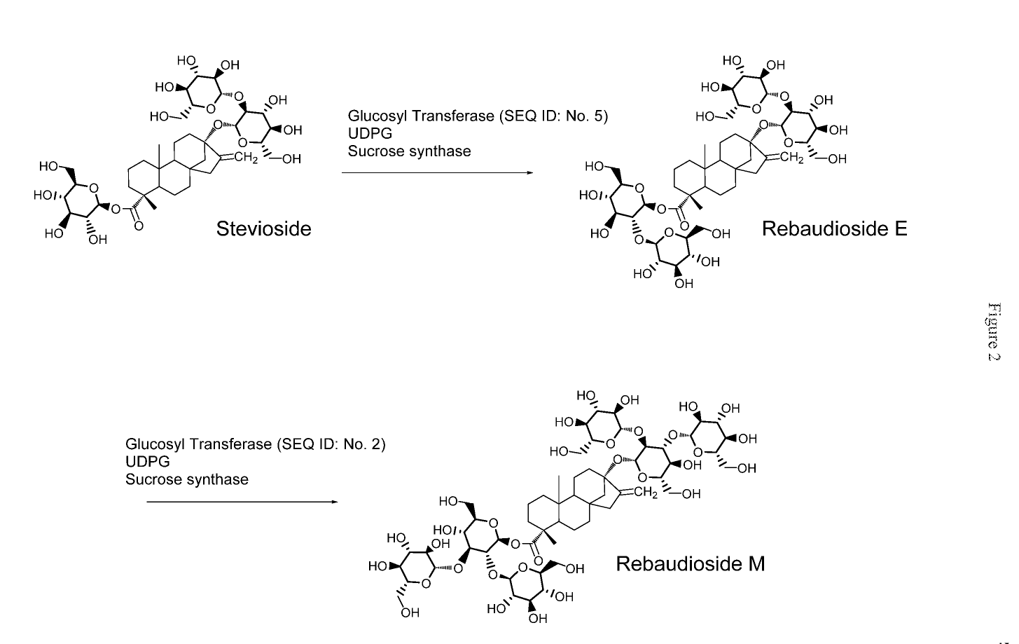

Embodiments of the method of producing Reb M are illustrated schematically in FIGS. 1-4.

Scheme 1 (FIG. 1) shows how Reb A is converted to Reb M. First, Reb A is converted to rebaudioside D (Reb D) by the Hordeum vulgare glucosyltransferase (SEQ ID NO:5). Reb D is then converted to Reb M by the Stevia rebaudiana glucosyltransferase UGT76G1 (SEQ ID NO:2).

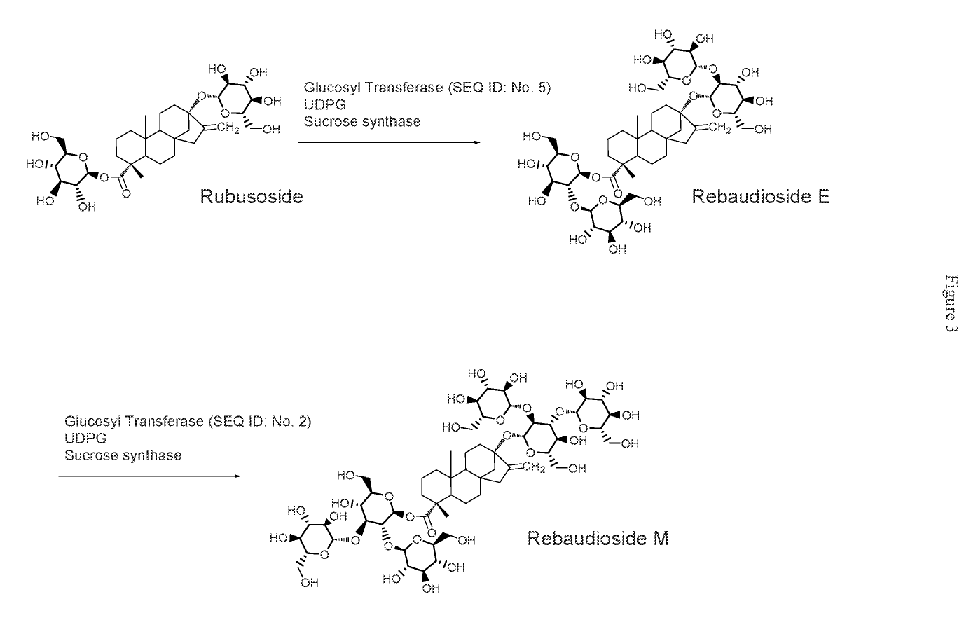

Scheme 2 (FIG. 2) shows how stevioside is converted to Reb M. First, stevioside is converted to rebaudioside E (Reb E) by the Hordeum vulgare glucosyltransferase (SEQ ID NO:5). Reb E is then converted to Reb M by the Stevia rebaudiana glucosyltransferase UGT76G1 (SEQ ID NO:2), which catalyzes the simultaneous addition of two glucose units to Reb E, one at C-19 hydroxyl and one at C-13 ester group, respectively.

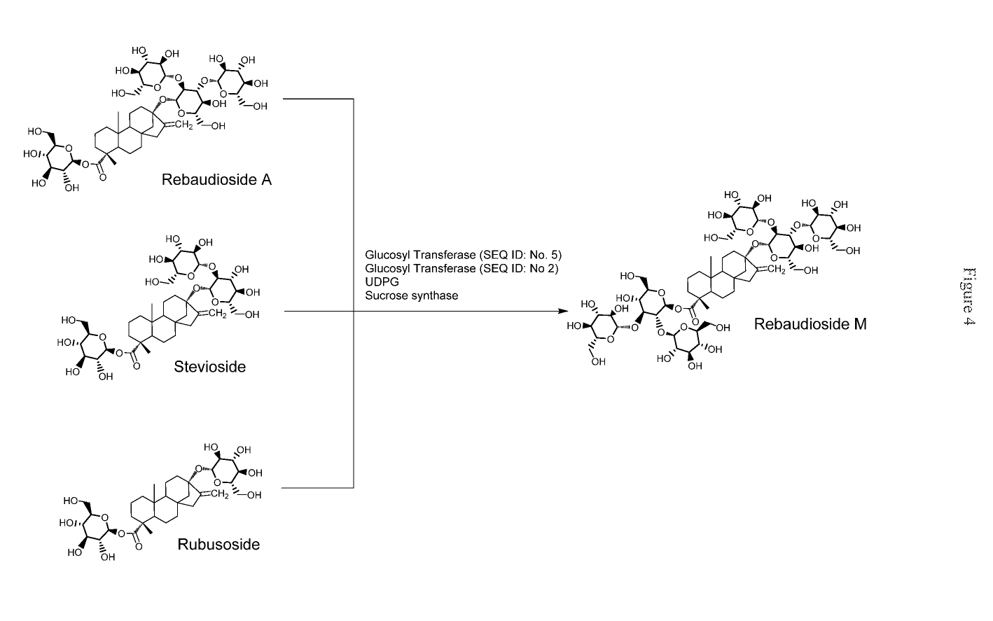

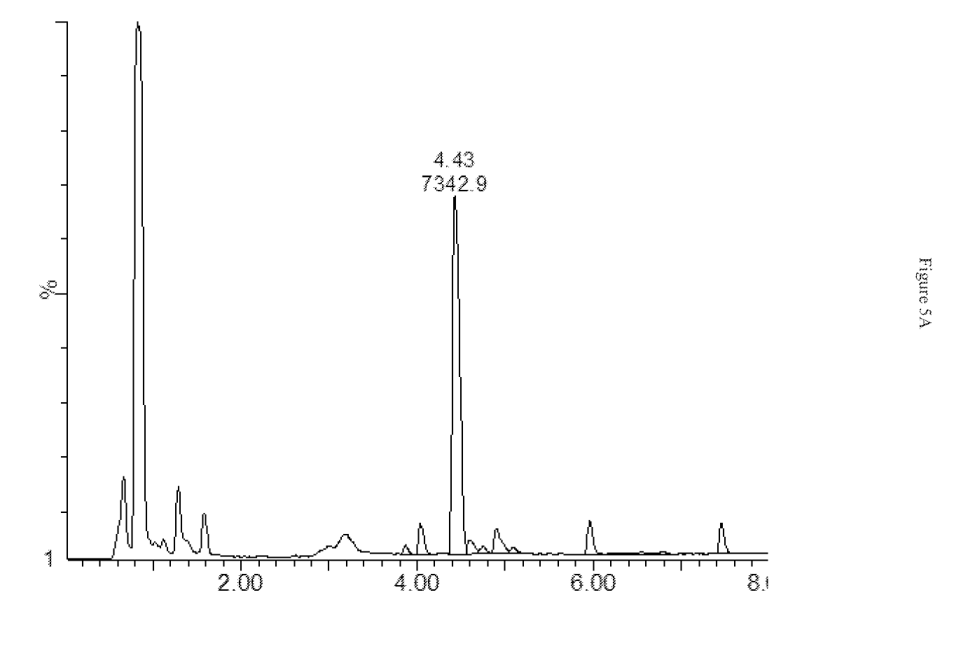

Scheme 3 (FIG. 3) shows how Rubusoside is converted to Reb M. First, Rubusoside is converted to Reb E by the Hordeum vulgare glucosyltransferase (SEQ ID NO:5). Reb E is converted to Reb M by the Stevia rebaudiana glucosyltransferase UGT76G1 (SEQ ID NO:2), which catalyzes the simultaneous addition of two glucose units to Reb E, one at C-19 hydroxyl and one at C-13 ester group, respectively.

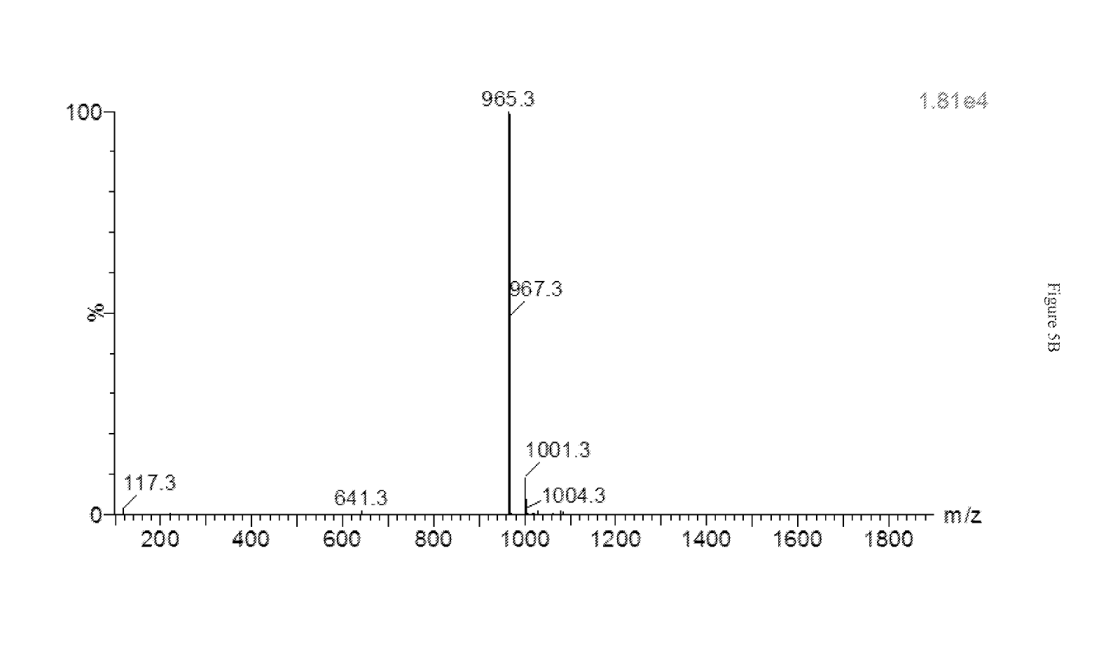

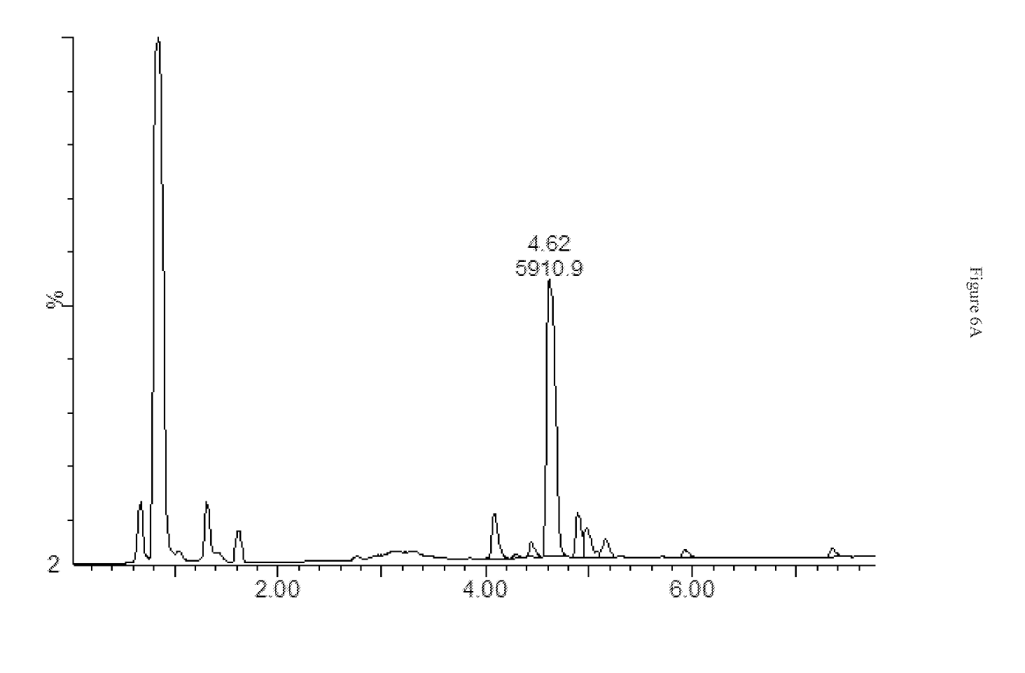

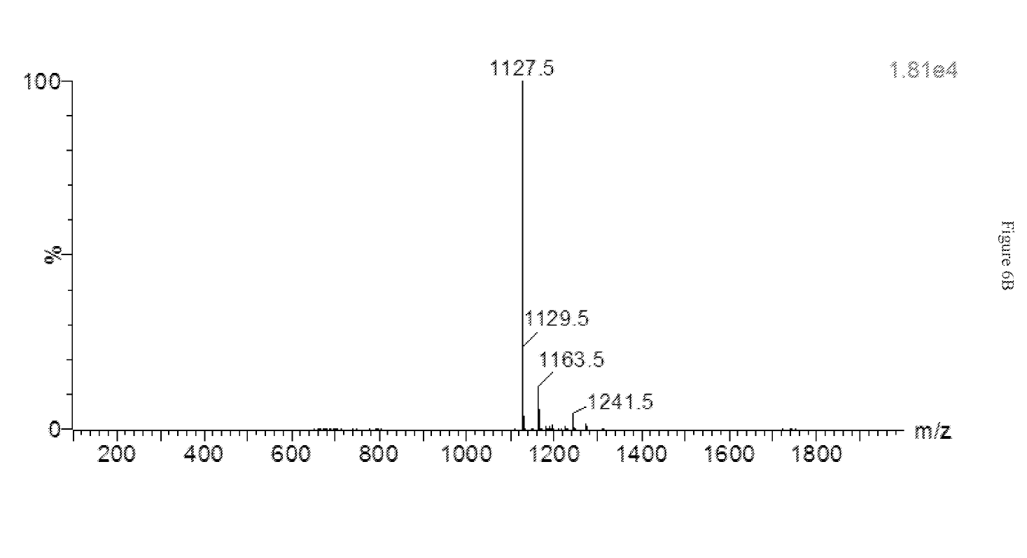

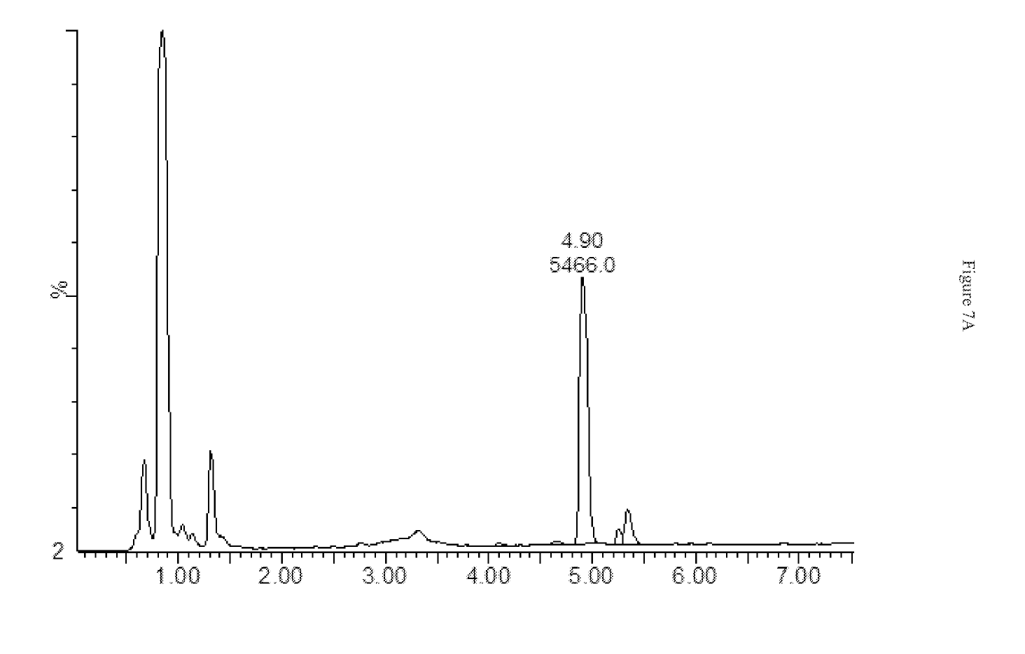

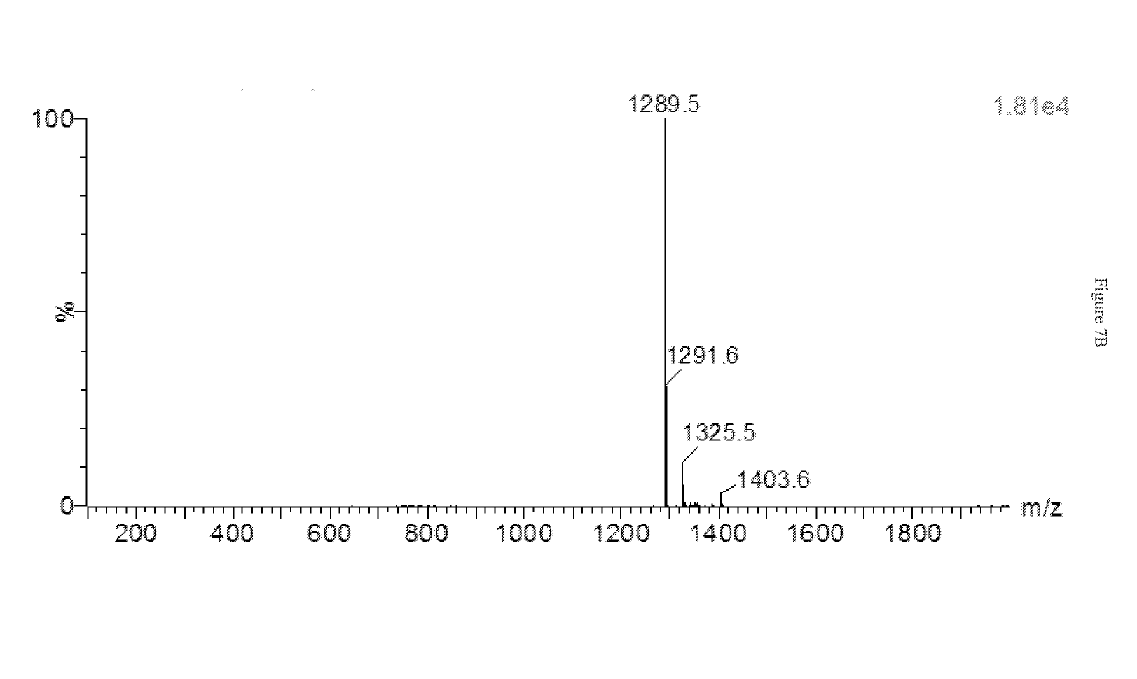

Scheme 4 (FIG. 4) shows how the sequential reactions illustrated in Scheme 1, 2, or 3 can be carried in one reaction vessel. That is, Reb M is the major product when at least one of rubusoside, stevioside or rebaudioside is incubated simultaneously with both the H. vulgare glucosyltransferase (SEQ ID NO:5) and the Stevia rebaudiana glucosyltransferase UGT76G1 (SEQ ID NO:2) in the presence of a UDPG re-generating system comprising sucrose synthase and UDPG.

The Reb M produced according to the disclosed methods can be further purified and used, for example, in products such as food, beverages, and pharmaceutical compositions.

Each reference cited in this disclosure is incorporated herein in its entirety. The following examples illustrate but do not limit the scope of the disclosure set forth above.

### Example 1: Construction of the Stevia Glucosyltransferase Expression Vector

In order to achieve highly efficient expression of the Stevia glucosyltransferase UGT76G1 in E. coli, the polynucleotide sequence (SEQ ID NO:1) encoding UGT76G1 (SEQ ID NO:2) was subjected to rare codon mutation. The resulting nucleotide sequence (SEQ ID NO:3) was obtained using total gene synthesis methods and cloned in the NdeI and XhoI sites of the pET-30a expression vector to obtain an expression plasmid for UGT76G1, which was called pNYK-C1. The plasmid pNYK-C1 was then transformed into E. coli Bl21 (DE3) by standard methods.

### Example 2: Construction of the Barley Glucosyltransferase Expression Vector

The polynucleotide sequence (SEQ ID NO:4) encoding the glucosyltransferase C5 from barley (Hordeum vulgare subsp. Vulgare) (SEQ ID NO:5) was subjected to rare codon mutation. The resulting polynucleotide sequence (SEQ ID NO:6) was obtained using total gene synthesis methods and cloned in the NdeI and XhoI sites of the pET-30b expression vector to obtain an expression plasmid for glucosyltransferase C5 from barley, which was called pNYK-05. The pNYK-05 plasmid was then transformed into E. coli Bl21 (DE3) by standard methods.

### Example 3: Preparation of Stevia UGT76G1 Crude Enzyme Solution

A pNYK-C1 clone was selected, transferred to 200 ml LB culture medium, and incubated at 37° C. overnight. Two ml of the resulting culture was transferred to 2000 ml of sterilized culture medium containing 10 g/L tryptone, 5 g/L yeast extract, 3.55 g/L disodium hydrogen phosphate, 3.4 g/L potassium dihydrogenphosphate, 2.68 g/L ammonium chloride, 0.71 g/L sodium, 0.493 g/L magnesium sulfate heptahydrate, 0.027 g/L ferric chloride hexahydrate, 5 g/L glycerol, 0.3 g/L glucose, and 50 mg/L kanamycin. The resulting solution was left at 37° C. until it reached an OD of 1.5-2. The conical flask was then placed immediately in a shaker with 300 rpm at 25° C. for 1 hr. IPTG was then added to culture with the final concentration of 0.5 mM. The resulting culture was left in the shaker set at 300 rpm at 25° C. After shaking at the same conditions for 16 hrs, the culture was cooled to 4° C., then centrifuged at 6,000×g for 20 min to obtain a wet cell mass of approximately 32 g. The precipitate was washed twice with distilled water to collect the transformed cells, which were then resuspended in 64 ml of distilled water and ice mixture. The resulting cell suspension was broken using a sonicator for 2 hrs to give ˜100 ml of a crude solution of Stevia UGT76G1.

### Example 4: Preparation of a Crude Sucrose Synthase Enzyme Preparation (“C4”)

A polynucleotide sequence (SEQ ID NO:7) encoding sucrose synthase from Arabidopsis thaliana (SEQ ID NO: 8) was synthesized and cloned into the pET30a expression vector, which was then transformed into E. coli BL2(DE3) as described in Example 1. A crude sucrose synthase enzyme solution (“C4”) was prepared as described in Example 3.

### Example 5: Preparation of a Crude Barley Glucosyltransferase Enzyme Preparation (“C5”)

A crude Barley glucosyltransferase enzyme preparation (“C5”) was obtained according to the procedure in Example 3.

### Example 6: Biotransformation of Stevioside to Reb E Using the Crude Enzyme Preparations C4 and C5

To the mixture containing stevioside (2.4 mM), UDPG (0.6 mM), TrisHCl buffer (100 mM, pH 8.0), and sucrose (0.1M) was added 0.21 ml of the crude C4 enzyme preparation and 1.26 ml of the crude C5 enzyme preparation, and water was then added to adjust the final volume to 3.7 ml. The resulting solution was shaken at 300 rpm at 30° C. After shaking at the same temperature for 24 hrs, the reaction mixture was heated to 90° C. for 20 min, centrifuged to obtain the supernatant, and then aliquoted for LCMS analysis. The retention times for stevioside and Reb E were 5.97 and 4.05, respectively. An authentic analytical sample of Reb E was purchased from Chromadex Inc, Irvine, Calif. and used as a LCMS standard reference. The results indicated that all of the starting stevioside was consumed in the reaction, with 65% being biotransformed to Reb E.

### Example 7: Optimization of the Conditions for Biotransformation of Stevioside to Reb E Using the Crude Enzyme Preparation C5

### Optimization Example 1, Reduction of Side Products

To a solution of stevioside (2.4 mM), UDPG (0.6 mM), TrisHCl buffer (100 mM, pH 8.0), and sucrose (0.1M) was added crude C5 enzyme solution (0.21 ml) and crude C4 enzyme solution (0.21 ml). Water was added to adjust the final volume to 3.7 ml. The resulting mixture solution was placed in a shaker and shaken at 300 rpm at 30° C. After shaking at the same conditions for 24 hrs, the reaction was heated to 90° C. for 20 min, then centrifuged to obtain the supernatant and aliquoted for LCMS. Results indicated the biotransformation of stevioside to Reb E is 90%.

### Optimization Example 2, Increased Substrate Concentration

The method of Example 6 was repeated except that the stevioside concentration was increased to 5 mM. Results indicated the biotransformation of stevioside to Reb E is 96%.

### Optimization Example 3, Reduction in the Amount of UDPG

The method of Example 6 was repeated, with the following modifications: the stevioside concentration was 2.4 mM, the UDPG concentration was 0.08 mM, 2.52 ml of the C5 enzyme solution was used. Results indicated the biotransformation of stevioside to Reb E is 85%.

### Optimization Example 4, without Sucrose Synthase

The method of Optimization Example 3 was repeated in the absence of sucrose synthase. To a solution of stevioside (5 mM), UDPG (4.8 mM), TrisHCl buffer (100 mM, pH 8.0), and sucrose (0.1M) was added crude C5 enzyme solution (2.52 ml) and crude C4 enzyme solution (0.21 ml), respectively. Water was added to adjust the final volume to 3.7 ml. The resulting mixture solution was placed in a shaker and shaken at 300 rpm at 30° C. After shaking at the same conditions for 24 hrs, the reaction was heated to 90° C. for 20 min, then centrifuged to obtain the supernatant and aliquoted for LCMS. Results indicated the biotransformation of stevioside to Reb E is 68%.

### Example 9: Biotransformation of Rubusoside to Rebaudioside E Using the Crude Enzyme Preparation C5

To a solution of rubusoside (5 mM), UDPG (0.08 mM), TrisHCl buffer (100 mM, pH 8.0) and sucrose (0.1M) was added crude C5 enzyme solution (2.52 ml) and crude C4 enzyme solution (0.21 ml), respectively. Water was added to adjust the final volume to 3.7 ml. The resulting mixture solution was placed in a shaker and shaken at 300 rpm at 30° C. After shaking at the same conditions for 24 hrs, the reaction was heated to 90° C. for 20 min, then centrifuged to obtain the supernatant and aliquoted for LCMS. Results indicated the biotransformation of stevioside to Reb E is 85%.

### Example 10: Biotransformation of Reb A to Reb D Using the Crude Enzyme Preparation C5

To solution of Reb A (5 mM), UDPG (0.08 mM), TrisHCl buffer (100 mM, pH 8.0) and sucrose (0.1M) was added crude C5 enzyme solution (2.52 ml) and crude C4 enzyme solution (0.21 ml), respectively. Water was added to adjust the final volume to 3.7 ml. The resulting mixture solution was placed in a shaker and shaken at 300 rpm at 30° C. After shaking at the same conditions for 24 hrs, the reaction was heated to 90° C. for 20 min, then centrifuged to obtain the supernatant and aliquoted for LCMS. Results indicated the biotransformation of Reb A to Reb D is 85%.

### Example 11: Biotransformation of Reb E to Reb M Using the Crude Enzyme Preparation C1

This example illustrate the continuous sequential biotransformation of stevioside first to Reb E, then Reb M. The crude reaction mixture from example was used directly for the preparation of Reb M. To the mixture of UDGP (0.6 mM), TrisHCl buffer (100 mM, pH 8.0) and sucrose (0.1 M) was added the supernatant (2.65 ml) from example 6 and the crude C1 enzyme preparation (2.38 ml). Water was then added to adjust the final volume to 6 ml. The resulting mixture solution was placed in a shaker and shaken at 300 rpm at 30° C. After shaking at the same conditions for 24 hrs, the reaction was heated to 90° C. for 20 min, then centrifuged to obtain the supernatant and aliquoted for LCMS. Results indicated the biotransformation of Reb E to Reb M was 68%. Retention time for Reb M is 4.87, an authentic analytical sample of Reb M was purchased from Chromadex Inc., Irvine, Calif. and used as a LCMS reference standard.

### Example 12: Biotransformation of Reb D to Reb M Using the Crude Enzyme Preparation C1

This example illustrates the continuous sequential biotransformation of Reb A first to Reb D, then to Reb M using the crude enzyme preparation C1. To the mixture of UDGP (0.6 mM), TrisHCl buffer (100 mM, pH 8.0) and sucrose (0.1 M) was added the supernatant (2.65 ml) from example 6 and the crude C1 enzyme preparation (2.38 ml). Water was then added to adjust the final volume to 6 ml. The resulting mixture solution was placed in a shaker and shaken at 300 rpm at 30° C. After shaking at the same conditions for 24 hrs, the reaction was heated to 90° C. for 20 min, then centrifuged to obtain the supernatant and aliquoted for LCMS. Results indicated the biotransformation of Reb D to Reb M was 95%. Retention time for Reb M is 4.87, an authentic analytical sample of Reb M was purchased from Chromadex Inc., Irvine, Calif. and used as a LCMS reference standard.

### Example 13: One Vessel Biotransformation of Rubusoside to Reb M Using Crude Enzyme Preparations C1 and C5

This example illustrates the biotransformation of rubusoside to Reb M in one reaction vessel.

To a solution of rubusoside (5 mM), UDPG (0.08 mM), TrisHCl buffer (100 mM, pH 8.0) and sucrose (0.1M) was added 2.52 ml of crude enzyme solution C5, 2.38 ml of crude enzyme solution C1, and 0.21 ml of crude enzyme solution C4 (0.21 ml). Water was added to adjust the final volume to 3.7 ml. The resulting mixture solution was placed in a shaker and shaken at 300 rpm at 30° C. After shaking at the same conditions for 24 hrs, the reaction was heated to 90° C. for 20 min, then centrifuged to obtain the supernatant and aliquoted for LCMS. Results indicated the biotransformation of stevioside to Reb M is 90%.

### Example 14: Biotransformation of Stevioside to Reb M in a Single Reaction Vessel Using Crude Enzyme Preparations C1 and C5

This example illustrate the biotransformation of stevioside to Reb M in a single reaction vessel.

To a solution of stevioside (5 mM), UDPG (0.08 mM), TrisHCl buffer (100 mM, pH 8.0) and sucrose (0.1M) was added the crude enzyme solution of C5 (2.52 ml), C1 (2.38 ml) and C4 (0.21 ml), respectively. Water was added to adjust the final volume to 3.7 ml. The resulting mixture solution was placed in a shaker and shaken at 300 rpm at 30° C. After shaking at the same conditions for 24 hrs, the reaction was heated to 90° C. for 20 min, then centrifuged to obtain the supernatant and aliquoted for LCMS. Results indicated the biotransformation of stevioside to Reb M is 85%.

### Example 15: Biotransformation of Reb A to Reb M in a Single Reaction Vessel Using Crude Enzyme Preparations C1 and C5

This example illustrate the biotransformation of Reb A to Reb M in a single reaction vessel.

To a solution of Reb A (5 mM), UDPG (0.08 mM), Tris HCl buffer (100 mM, pH 8.0) and sucrose (0.1M) was added the crude enzyme solution of C5 (2.52 ml), C1 (2.38 ml) and C4 (0.21 ml), respectively. Water was added to adjust the final volume to 3.7 ml. The resulting mixture solution was placed in a shaker and shaken at 300 rpm at 30° C. After shaking at the same conditions for 24 hrs, the reaction was heated to 90° C. for 20 min, then centrifuged to obtain the supernatant and aliquoted for LCMS. Results indicated the biotransformation of stevioside to Reb M is 90%.

1. A method of producing rebaudioside M (Reb M), comprising incubating in a single reaction vessel under reaction conditions suitable for glucosyltransferase activity, wherein the reaction conditions comprise a pH of 7.0-9.0 and a temperature of 20° C.-37° C.:
(a) at least one rebaudioside selected from the group consisting of rebaudioside A (Reb A), stevioside, and rubusoside;
(b) a glucosyltransferase comprising the amino acid sequence SEQ ID NO:5;
(c) a glucosyltransferase comprising the amino acid sequence SEQ ID NO:2;
(d) uridine diphosphate glucose (UDPG); and
(e) sucrose synthase,
thereby producing Reb M.

2. The method of claim 1, wherein the single reaction vessel comprises at least two rebaudiosides selected from the group consisting of Reb A, stevioside, and rubusoside.

3. The method of claim 1, wherein the single reaction vessel comprises Reb A, stevioside, and rubusoside.

4. The method of claim 1, wherein the reaction conditions comprise pH 8.0.

5. The method of claim 1, wherein the reaction conditions comprise 30° C.

6. The method of claim 4, wherein the reaction conditions comprise 30° C.

7. The method of claim 2, wherein the reaction conditions comprise pH 8.0.

8. The method of claim 2, wherein the reaction conditions comprise 30° C.

9. The method of claim 7, wherein the reaction conditions comprise 30° C.

10. The method of claim 3, wherein the reaction conditions comprise pH 8.0.

11. The method of claim 3, wherein the reaction conditions comprise 30° C.

12. The method of claim 10, wherein the reaction conditions comprise 30° C.
